(19) United States
(12) Patent Application Publication
(10) Pub. No.: US 2020/0376001 A1
(43) Pub. Date: Dec. 3, 2020

(54) USE OF ANDROGEN FOR TREATING GASTROINTESTINAL DISORDERS

(71) Applicant: Edward M. Lichten, Birmingham, MI (US)

(72) Inventor: Edward M. Lichten, Birmingham, MI (US)

(21) Appl. No.: 16/997,408

(22) Filed: Aug. 19, 2020

Related U.S. Application Data
(63) Continuation-in-part of application No. 15/456,816, filed on Mar. 13, 2017, now abandoned.
Application No. 15/456,816 is a continuation-in-part of application No. 14/035,237, filed on Sep. 24, 2013, now abandoned.
(60) Provisional application No. 61/704,797, filed on Sep. 24, 2012.
(60) Provisional application No. 61/704,797, filed on Sep. 24, 2012.

(51) Int. Cl. A61K 31/568; A61K 31/5685; A61K 31/58; A61K 9/00

(52) U.S. Cl. CPC A61K 31/568; A61K 31/5685; A61K 9/0053; A61K 9/0019; A61K 31/58

(57) ABSTRACT

By initially lowering the SHBG with a synthetic androgen and, thereafter, raising the testosterone level in a person's body, an effective method for treating gastrointestinal disorders such as irritable bowel syndrome, inflammatory bowel diseases, including Crohn's Disease and ulcerative colitis, is achieved by first using a synthetic anabolic steroid and, thereafter, a natural androgen.

### CROSS-REFERENCE TO RELATED APPLICATION

This application is a continuation-in-part application which claims the priority benefit of co-pending U.S. patent application Ser. No. 15/456,816, filed Mar. 13, 2017, for “Use of Androgen For Disease Treatment,” which claims the priority benefit of U.S. patent application Ser. No. 14/035,237, filed Sep. 24, 2013, which claims the priority benefit of U.S. Provisional Patent Application Ser. No. 61/704,797, filed Sep. 24, 2012, the entire disclosures of which are hereby incorporated by reference in their entirety.

### BACKGROUND OF THE INVENTION

### 1. Field of the Invention

The present invention pertains to a method for treating gastrointestinal disorders including irritable bowel syndrome and inflammatory bowel disease. More particularly, the present invention pertains to a method for overcoming exposure to environmental toxins causing gastrointestinal disorders. Even more particularly, the present invention pertains to the use of androgens for lowering the level of Sexual Hormone Binding Globulin (SHBG), reducing elevated cytokines normalizing estrogen receptor beta blunting the abnormal inflammatory response to disease in a patient to achieve a suitable Free Androgen Index (FAI) which correlates to effectively treating gastrointestinal disorders.

### 2. Description of Related Art

As is known to those of ordinary skill in the art to which the present invention pertains, there has been reported the use of an androgen, such as stanozolol, for blocking liver production of SHBG. Further, the prior art is replete with reports showing the use of the laboratory measurement of SHBG using an androgen as a marker, sensitivity test, androgen receptor, mutation detection, and so forth. Furthermore, U.S. Pat. Nos. 7,186,706, 6,583,129, and 6,139,873 illustrate the utilization of stanozolol and other androgens in connection with SHBG testing.

Heretofore, toxins, including organic, genetic, environmental, and so forth, have been the subject of many hypotheses in studies determining the cause of gastrointestinal disorders within the gastrointestinal (GI) tract and, in particular, IBD.

The GI tract includes all structures between the mouth and the anus, which forms a continuous passageway including the main organs of digestion, namely, the stomach, small intestine, and large intestine.

Common infections and diseases resulting from overexposure of these environmental toxins include irritable bowel syndrome (IBS) and inflammatory bowel disease (IBD).

IBS is a functional gastrointestinal disorder and includes symptoms such as abdominal pain and changes in the pattern of bowel movements without any evidence of underlying damage. IBS has been classified into four different categories based upon whether diarrhea is common (IBS-D), constipation is common (IBS-C), both are common (IBS-M), or neither occurs very often (IBS-U).

IBS is commonly treated by altering one's diet to avoid certain carbohydrates or consume more fiber, taking medication such as laxatives or those increasing the amount of serotonin in the intestines, or taking probiotics. Oftentimes, IBS symptoms can sometimes be controlled, using any one or combination of the above treatment methods.

IBD is an autoimmune disease where the body's own immune system attacks elements of the digestive system. IBD primarily includes Crohn's disease and ulcerative colitis. Both Crohn's disease and ulcerative colitis are oftentimes difficult to distinguish from one another as they both present similar symptoms, for example, abdominal pain, vomiting, diarrhea, rectal bleeding, severe internal cramps/muscle spasms in the pelvis region, and weight loss. Nonetheless, the main difference between the two diseases is the location of the inflammatory changes. Crohn's may affect any part of the GI tract while ulcerative colitis is restricted to the colon and the rectum. Today, each is treated in the same manner, i.e., AZA Methotrexate, DMARDS, biologics and surgery.

Treatment of ulcerative colitis may include surgery where the rectum and colon is removed. This results in the patient having to use an ileostomy bag to collect feces. Crohn's disease, on the other hand, cannot be cured by surgery. Instead, surgery is used to remove the inflamed parts of the intestines to treat complications such as abscesses, strictures, or fisulae.

As is disclosed hereinafter, the predicate for the present invention resides in the postulate that environmental toxins cause gastrointestinal disorders and these toxins affect the body with respect to the levels of SHBG. Therefore, by blocking or lowering the level of SHBG and subsequently raising the testosterone level, the FAI equilibrium in the body is changed, thereby reducing the effects of the environmental toxins on pituitary hormones, gonadal hormones, and intracellular estrogen receptors and cytokines and minimizing the effects of these gastrointestinal disorders.

In essence, by utilizing an androgen to lower the SHBG in the body, there is a decrease in the effect of the endocrine disruption compounds, thereby reducing the propensity of the body to become diseased.

More particularly, by reducing the effects of these environmental toxins with regards to lowering the SHBG in the body, a gastrointestinal disorder may be treated, and side effects minimized.

It is to this to which the present invention is directed.

### SUMMARY OF THE INVENTION

In accordance with the present invention, there is provided a method for treating gastrointestinal disorders, specifically, IBS and IBD, including Crohn's disease and ulcerative colitis.

The present method comprises the steps of lowering the SHBG in the body through the utilization of a synthetic androgen and, thereafter, raising the serum testosterone level in the body through the use of a natural androgen to raise the FAI to an acceptable level.

More specifically, by utilizing selected amounts of synthetic androgens and the second natural androgens, it is possible to first lower the amount of SHBG in the body with the synthetic androgen and, once the reduced level is achieved, raising the testosterone level in the body with the natural androgen to achieve a specified FAI and an equilibrium between the SHBG and the natural androgen.

Preferably, the androgens used herein are steroids. Both naturally occurring and synthetic steroids may be used.

For a more complete understanding of the present invention, reference is made to the following detailed description of the invention.

### DETAILED DESCRIPTION OF THE INVENTION

As noted above, the present invention provides a method for treating gastrointestinal disorders such as IBS and IBD, including Crohn's disease and ulcerative colitis.

To avoid having to resort to surgery or alternative medications commonly employed to treat IBS and IBD, the present method comprises the steps of, first, lowering the SHBG in the body through the utilization of a first androgen and, thereafter, once the SHBG reaches the desired reduced level, utilizing a second androgen to raise the serum testosterone level, thereby reaching an acceptable FAI to manage symptoms of gastrointestinal disorders.

As is known to one of ordinary skill in the art, the FAI in a person is determined by a ratio of the serum testosterone level with respect to the SHBG in that person. In other words, FAI=testosterone level/SHBG. Thus, the severity of gastrointestinal disorders exhibited by IBD and IBS is an inverse relationship between the amount of SHBG in comparison to the serum testosterone level in a patient.

Therefore, by first lowering the amount of SHBG with the synthetic androgen and, then, adding the natural androgen into the body to raise the serum testosterone level, a desired increased FAI may be reached and the effective treatment of gastrointestinal disorders caused by environmental toxins is substantially increased.

More specifically, by utilizing selected amounts of the synthetic androgen, it is possible to lower the amount of SHBG in the body up to 8 fold.

Useful synthetic androgens include stanozolol and/or danazol which are to lower the SHBG by 80% and lowers the testosterone or FAI.

Generally, the synthetic androgen is administered over a period of time of about four to about eight weeks in order to lower the SHBG to within a young adult range of about 30 isomole per liter (nmol/l) to about 45 nmol/l for a female and to within a range for an older male of about 5 nmol/l to about 15 nmol/l.

After the treatment with the synthetic androgen which lowers the total testosterone concentration to a specified range, it is necessary to, then, add the natural androgen into the system to raise the serum testosterone to a normal level and to raise the FAI in the individual to an acceptable level.

In practicing the present invention, both natural and synthetic androgens may be used.

Representative natural androgens, used herein are, for example, dihydroepiandrosterone, androstenedione, androstenediol, androsterone, dihydrotestosterone, testosterone, nandrolone, and the like.

Synthetic androgens used herein include in addition to the stanozolol and danazol, for example, oxandrin, furzabolin, dianabol, anadrol, and the like.

As noted, in the practice hereof, the synthetic androgen which is a synthetic anabolic steroid is used to lower the SHBG concentration. The synthetic anabolic steroids include, for example: stanozolol, dianabol, danazol, and anadrol.

Preferably, stanozolol is used as the synthetic androgen for lowering the SHBG. The stanozolol can be administered either orally or as an injectable.

The gender of the patient or person determines the amount of natural anabolic steroid necessary to raise the testosterone level in order to achieve the desired FAI. For a male, the FAI should range from about 1 to about 2. For a female, the FAI should range from about 0.03 to about 0.10. Thus, a male needs to have an SHBG level of from about 5 nmol/l to about 15 nmol/l and a female from about 30 nmol/l to about 45 nmol/l in order to ultimately achieve the desired FAI.

Regardless of gender, generally, from about 4.0 milligrams (mgs) to about 8.0 mgs of the stanozolol is administered orally, daily, for about four to about eight weeks in order to effectuate specific lowering of the SHBG.

Where injections of the natural androgen are used, generally, from about 10.0 mgs to about 50.0 mgs are injected, once a week, for about four to about eight weeks.

As noted above, after the SHBG has been lowered with the synthetic androgen to an acceptable level, the natural androgen, such as an anabolic steroid, is, then, administered into the body to rebalance the equilibrium in the body to raise the serum testosterone and the FAI.

Where the patient is a male, generally from about 100 mgs to about 250 mgs of “added back” natural androgen, e.g., testosterone is administered as an injection, once a week, for about four to about eight weeks, at which time the dosage is modified to about 5 to 10 mgs to reduce side effects and optimize clinical presentation.

With a female patient, the amount of natural anabolic testosterone administered will generally range from about 15 mgs to about 70 mgs, once a week, for about four to about eight weeks.

According to the present method where the testosterone and nandralone are used in combination, generally, they will be administered in a respective weight ratio of from about 4.0:1.0 to about 1.0:1.0 of testosterone to nandralone.

The administration of the first and second androgens may be continued indefinitely as the desired levels of FAI is achieved, Preferably, periodic measurements of SHBG and testosterone levels in the patient are taken in order to determine if the desired ranges have been achieved. It is to be understood that it is well known to those of ordinary skill in the art to measure SHBG and testosterone levels in a patient using any standard blood or saliva test.

By following the steps described herein, generally, in about four to eight weeks, equilibrium between the SHBG and the testosterone is achieved, and the FAI will fall within the desired range, i.e., FAI is greater than 1. It is to be appreciated that by administering the specified amounts of androgen, described above, there is a concomitant “re-balancing” of an appropriate testosterone level in relation to the decrease in SHBG.

Although not wishing to be bound by any theory, it appears that the first or the initial insult on the immune system is the environmental toxin/xenoestrogen presence. It is a stressor, just like a viral infection that triggers an adrenal response. The body's General Adaptation Syndrome causes an outpouring of corticosteroids from the Hypothalamic-Pituitary-Adrenal Axis (HPA). This suppresses the Hypothalamic-Pituitary-Gonadal Axis (HPG) that produces testosterone.

Concurrently, estrogens and specifically xenoestrogens increase the liver production of Sex Hormone Binding Globulin (SHBG). As reported by Anderson in 1972 SHBG binds 98% of testosterone. Therefore, there is both a decrease in testosterone and an increase in SHBG. The Free Androgen Index [TT/SHBG] is the best simple readily available measurement/Laboratory Test of what is hormonally occurring.

The use of the natural androgen is directed to the loss of bioavailable testosterone [TT/SHBG]. This reduces testosterone binding to the outside of a cell (Androgen Receptor). Therefore, with testosterone binding to the cell, the testosterone present in cytoplasma is reduced and its ability to bind to the estrogen receptors on the nuclear membrane is reduced.

The addition of stanozolol decreases SHBG by about 80%, thereby increasing the FAI. Nandrolone is only 5% bonded to SHBG but has a greater affinity for SHBG by about three times than that of testosterone. Nandrolone frees testosterone from SHBG. Thus, the combination of natural and synthetic androgens raises the FAI bioavailable testosterone which stabilizes the Estrogen Receptor Beta.

It is contemplated that by employing the above method to reduce high levels of SHBG in a person's body and, thereafter, raise the serum testosterone level, a satisfactory FAI can be achieved, thereby treating IBS and IBD as FAI represents available testosterone in the cascade from disease to autoimmune response. As a result thereof, symptoms and side effects of gastrointestinal disorders may be managed and minimized.

Stanozolol reduces the serum concentration of SHBG by 80%. Danazol does this as well. Stanozolol is 100 times more potent mg for mg than danazol. Using stanozolol reduces side effects and hirsutism caused by danazol.

The FAI in men is typically increased 4 fold; in women it can increase by 20 fold.

It is to be understood that the androgens can be administered substantially simultaneously or sequentially of synthetic androgen followed by the natural androgen.

It is to be understood that the present invention is not for measuring the serum TT/SHBG biomarkers as the FAI, but for a method of treatment directly attributed to changes in the intracellular levels/ratio of the Estrogen Receptors Beta that cannot be easily measured and is both time consuming and extremely expensive.

The estrogen receptor beta to alpha receptor ratio is a biomarker for indicating inflammation in the gastrointestinal system as well as for autoimmune diseases. Therefore, it is believed that the corollary is that when the immune system is not in hemostasis that the FAI is suppressed. The FAI drives the immune system to hemostasis.

Having thus described the invention, what is claimed is:

1. A method for treating gastrointestinal disorders, comprising the steps of:
(a) administering to a person a predetermined amount of a synthetic androgen over a period of time of from about four to about eight weeks to lower the SHBG level of the person to a pre-selected level;
(b) thereafter, administering a naturally occurring androgen to the person over a period of time of from about four to about eight weeks to raise the testosterone to a normal level of the person to achieve an acceptable Free Androgen Index (FAI) according to the gender of the person; and
wherein achieving an acceptable FAI minimizes the symptoms of a gastrointestinal disorder.

2. The method of claim 1 wherein the gastrointestinal disorder is irritable bowel syndrome.

3. The method of claim 1 wherein the gastrointestinal disorder is inflammatory bowel disease.

4. The method of claim 3 wherein the gastrointestinal disorder is Crohn's disease.

5. The method of claim 3 wherein the gastrointestinal disorder is ulcerative colitis.

6. The method of claim 3 wherein the synthetic and naturally occurring androgens are steroids.

7. The method of claim 6 wherein the naturally occurring androgen is selected from the group consisting of dihydroepiandrosterone, androstenedione, androstenediol, androsterone, dihydrotestosterone, testosterone, nandrolone, oxandrin, furazabol, dioxadol, and anadrol.

8. The method of claim 6 wherein the naturally occurring androgen is selected from the group consisting of testosterone and nandrolone.

9. The method of claim 8 wherein the naturally occurring androgen is nandrolone.

10. The method of claim 8 wherein the synthetic androgen is selected from the group consisting of danazol and stanozolol.

11. The method of claim 6 wherein the natural androgen is administered orally in an amount ranging from about 10 milligrams to about 8.0 milligrams daily for about four to eight weeks.

12. The method of claim 11 wherein the person is male, and the synthetic androgen is administered as an injection in an amount ranging from about 100 milligrams to about 250 milligrams, once a week, for about four to about eight weeks.

13. The method of claim 11 wherein the person is a female and the synthetic androgen is administered as an injection in an amount which ranges from about 15 milligrams to about 70 milligrams, once a week, for about four to about eight weeks.

14. The method of claim 3 wherein the SHBG is lowered to about 5 nanomoles per liter to about 15 nanomoles per liter for a male and to about 30 nanomoles per liter to about 45 nanomoles per liter for a female.

15. The method of claim 3 wherein the FAI after treatment ranges from about 1 to about 2 for a male and from about 0.03 to about 0.10 for a female.

16. The method of claim 1 which further comprises:
determining the estrogen receptor beta to alpha ratio as a biomarker for indicating an inflammation within the gastrointestinal tract prior to steps (a) and (b).

17. A method for treating Crohn's Disease, comprising the steps of:
(a) administering to a person a predetermined amount of a first natural androgen which is a natural anabolic steroid over a period of time of from about four to about eight weeks;
(b) thereafter, administering a second androgen which is a synthetic anabolic steroid to the person over a period of time of from about four to about eight weeks to raise the serum testosterone level of the person to achieve an acceptable Free Androgen Index (FAI) according to the gender of the person and lower the SHBG to within a range of about 30 nanomoles per liter (nmol/l) to about 45 nmol/l for a female and to within a range of about 5 nmol/l to about 15 nmol/l for a male to, thereby, minimize the symptoms of Crohn's Disease; and
(c) wherein:
(i) the first androgen is selected from the group consisting of dihydroepiandrosterone, androstenedione, androstenediol, androsterone, dihydrotestosterone, testosterone, nandrolone, oxandrin, furazabol, dioxadol, and anadrol, and the second androgen is selected from, and the second androgen is selected from the group consisting of stanozolol, dianabol, danazol and anadrol; and
(2) for a male, the acceptable FAI should range from about 1 to about 2 and for a female, the FAI should range from about 0.03 to about 0.10. to effectuate an SHBG level of from about 5 nmol/l to about 15 nmol/l for a male to from about 30 nmol/l to about 45 nmol/l in order to achieve the desired FAI:
and further wherein regardless of gender, generally, from about 4.0 milligrams to about 8.0 mgs of stanozolol is administered orally, as the second androgen for about four to about eight weeks in order to effectuate specific lowering of the SHBG; and
when injections are used from about 10.0 mgs to about 50.0 mgs are injected, once a week, for about four to about eight weeks; and when patient is a male, generally from about 100 mgs to about 250 mgs and where testosterone is administered as the second androgen as an injection, once a week, for about four to about eight weeks; and with a female patient, the amount of testosterone administered will range from about 15 mgs to about 70 mgs, once a week, for about four to about eight weeks.
